(19) United States
(12) Patent Application Publication
(10) Pub. No.: US 2024/0002773 A1
(43) Pub. Date: Jan. 4, 2024

(54) PLATE CLEANING DEVICE

(71) Applicant: HOFFMANN-LA ROCHE INC., Little Falls, NJ (US)

(72) Inventors: Edgar Ernesto CARRASCO, Basel (CH); Dieter SCHMIDLI, Basel (CH); Roger STEINER, Basel (CH); Joerg VOELKLE, Basel (CH); Johanna Alina SCHNEIDER, Penzber (DE)

(21) Appl. No.: 18/250,480

(22) PCT Filed: Nov. 5, 2021
(86) PCT No.: PCT/EP2021/080756

§ 371 Date: Apr. 25, 2023

(30) Foreign Application Priority Data
Nov. 6, 2020 (EP) 20206277.4

(51) Int. Cl. C12M 1/00; C12M 1/32; C12M 3/00; C12M 1/12

(52) U.S. Cl. CPC C12M 39/00; C12M 23/12; C12M 23/48; C12M 29/14; C12M 37/06

(57) ABSTRACT

A plate cleaning device (1) for cleaning a clamp plate of a fermenter, wherein the clamp plate has a plurality of receptacles each configured to hold a bioreactor vessel, and at least one conduit with a conduit opening per receptacle. The device comprises a plate seat member (3), a cover member (2), a plurality of valve units (4), a first fluid terminal (71), a second fluid terminal (74) and a control unit (9). The plate seat member (3) is configured to hold the clamp plate (6) in a predefined position and orientation. The cover member (2) is configured to close the plate seat member (3) such that the clamp plate (6) is tightly enclosed by the plate seat member (3) and the cover member (2). The plurality of valve units (4) is configured to be coupled to the cover member (2) such that at least one valve unit (4) is associated to each of the conduit openings (62) of the clamp plate (6). The first fluid terminal (71) is configured to receive a first fluid container and the second fluid terminal (74) to receive a second fluid container. The cover member (2) is equipped with channels each having a first open end and a second open end. Each of the first open ends of the channels of the cover member (2) is positioned to be connected to one of the conduit openings (62) of the clamp plate (6). The valve units (4) are configured to be coupled to the second open ends of the channels of the plate such that all of the at least one conduit openings (62) per receptacle are associated to one valve (4). The first fluid terminal (71) and the second fluid terminal (74) are coupled to each of the valve units (4). The control unit (9) is configured to selectively connect the first fluid terminal (71) and the second fluid terminal (74) to the second open ends of the channels of the cover member (2).

### TECHNICAL FIELD

The present invention is in the field of maintenance of fermentation apparatus and systems. More specifically, the present invention relates to cleaning a clamp plate of a fermenter, wherein the clamp plate has a plurality of receptacles each configured to hold a bioreactor vessel, and at least one conduit with a conduit opening per receptacle.

### BACKGROUND ART

For microbial or mammalian screening and optimization experiments it is known to use microscale bioreactors. In order to allow an efficient handling, monitoring and controlling of the experiments it is known to use specific systems including sophisticated apparatus, equipment and software. Particularly, in cases where a number of bioreactors are involved such systems allow for increasing efficiency and accuracy of the experiments.

For example, today the company Sartorius Stedim Biotech GmbH provides a widespread microbioreactor system which is traded under the brand Ambr® 15. This system involves a workstation and disposable (micro-) bioreactors which are (semi-) automatically processed. In particular, the system allows for applying, processing and controlling fermentation experiments in an at least partially automated manner.

Thereby, the systems of the art often use clamp plates for accurate processing of the bioreactors and their content. For example, the Ambr® 15 system may include ambr15cc clamp plates, i.e. type 001-0B38 clamp plates, having twelve receptacles each configured to hold one bioreactor, and plural conduits with a conduit opening per receptacle, or similar ambr15f clamp plates, i.e. type 001-3B38 clamp plates. The conduits allow for providing fluids to the bioreactor for various purposes.

Typically, in use the clamp plates of such systems get dirty or contaminated. However, cleaning usually is quite difficult, cumbersome and in some case dangerous since the conduits are comparably small such as of capillary dimension and since the used solvents may impair the health of an operator. Also, there typically is no feedback or information on the success of cleaning. Furthermore, in many experiments and particularly pharmaceutical experiments it is important to assure that the clamp plates are contamination free and that the conduits are free of full or partial blockage. Thereby, only minor residuals in the conduits of the clamp plates may impair future experiments or damage components of the system. Also, residuals may block or impair fluid flow which may hinder proper operation of the system or damage components of the system. Thus, the clamp plates typically are replaced after a certain number of uses, depending on the type of fermentation experiment performed even after comparably few uses such as after 20 use cycles, and/or after a certain time such as after one year of use.

However, clamp plates of systems of these types often are comparably expensive since they have to be manufactures at a comparably high accuracy. Thus, replacing the clamp plates may involve comparably high costs which lowers the economic efficiency of the systems. Moreover, inappropriately cleaned clamp plates may lead to a loss of the involved experiment due to blocked clamp plates. This may prevent meeting of timelines and cause considerable costs. Further, known cleaning procedures typically are cumbersome as they involve manual step and are not standardized or reproducible. Also, cleaning may cause aerosols which may harm persons. Therefore, there is a need for a system or device allowing to prepare clamp plates after use in a bioreactor system to increase their re-usability, avoid using dirty plates and/or prevent loss of time and resources. In particular, it may be desired to have a system or device processing clamp plates to achieve and guarantee sufficient cleanness such that they can be re-used in bioreactor systems.

### DISCLOSURE OF THE INVENTION

According to the invention this need is settled by a plate cleaning device as it is defined by the features of independent claim 1. Preferred embodiments are subject of the dependent claims.

In one aspect, the invention is a plate cleaning device for cleaning a clamp plate of a fermenter, wherein the clamp plate has a plurality of receptacles each configured to hold a bioreactor vessel, and at least one conduit with a conduit opening per receptacle. Typically, the clamp plate comprises a plurality of conduits such as one conduit per conduit opening connecting or associating the conduit opening with one of the receptacles. The plate cleaning device comprises a plate seat member, a cover member, a plurality of valve units, a first fluid terminal, a second fluid terminal and a control unit.

The plate seat member is configured to hold the clamp plate in a predefined position and orientation. The cover member is configured to close the plate seat member such that the clamp plate is tightly enclosed by the plate seat member and the cover member, when the plate seat member holds the clamp plate and the cover member closes the plate seat member.

The plurality of valve units is configured to be coupled to the cover member such that at least one valve unit is associated to each of the conduit openings of the clamp plate, when the plate seat member holds the clamp plate and the cover member closes the plate seat member.

The first fluid terminal is configured to receive a first fluid container and the second fluid terminal is configured to receive a second fluid container. The fluid containers may be any type of containers suitable for storing the respective fluid. They may be embodied as pressure or pressurized tanks or bottles, liquid tanks or vessels, or the like. The fluids may be any liquid or gas suitable for or assisting cleaning the clamp plate and particular its conduits.

The cover member is equipped with channels each having a first open end and a second open end. In particular, the open ends of the channels may open the channels towards an exterior of the cover member. Thereby, the channels may be accessed via the open ends.

Each of the first open ends of the channels of the cover member is positioned to be connected to one of the conduit openings of the clamp plate, when the plate seat member holds the clamp plate and the cover member closes the plate seat member. In an efficient implementation the cover member is embodied as cover plate. More specifically, the first open ends may be located and oriented at the cover member in correspondence with associated conduit openings of the clamp plate such that the channels of the cover member are in fluid communication with the conduit(s) of the clamp plate when the device houses the clamp plate.

The valve units are configured to be coupled to the second open ends of the channels of the plate such that all of the at least one conduit opening per receptacle are associated to one valve. The first fluid terminal and the second fluid terminal are coupled to each of the valve units.

The control unit is configured to selectively connect the first fluid terminal and the second fluid terminal to the second open ends of the channels of the cover member. Selectively connecting may cover any single one or sequence of, at least for a certain time, not connecting any of the first and second fluid terminals to the second open ends of the of the channels, exclusively connecting one of the first and second fluid terminals to the second open ends of the channels as well as connecting both of the first and second fluid terminals to the second open ends of the channels.

The plate cleaning device according to the invention allows for cleaning the clamp plate and particularly the conduit(s) thereof in a particularly sophisticated manner. By means of the specifically configured control unit, an appropriate sequence of the first and second fluids may be provided to the clamp plate at well monitored and adapted conditions. Like this, the clamp plate can efficiently and accurately be cleaned such that it can be re-used for a bioreactor or other similar system.

Preferably, the plate cleaning comprises a water tank as first fluid container, wherein the water tank is received in the first fluid terminal. In particular, the water tank can be received in the first fluid terminal by being detachably mounted to the first fluid terminal. For this, the first fluid terminal can be equipped with a seat into which the water tank can be put and a coupling structure configured to be connected to an outlet of the water tank.

When being equipped with the water tank, the plate cleaning device can provide water to the clamp plate for cleaning. In particular, the control unit can forward the water, e.g., cyclically through the conduit(s) of the clamp plate, e.g., for flushing. The water tank advantageously is filled with purified water which allows for an efficient cleaning.

Preferably, the plate cleaning device comprises an alcoholic liquid tank as second fluid container, wherein the alcoholic liquid tank is received in the second fluid terminal. In particular, the alcoholic liquid tank can be received in the second fluid terminal by being detachably mounted thereto.

When being equipped with the alcoholic liquid tank, the plate cleaning device can provide alcoholic liquid to the clamp plate for cleaning. In particular, the control unit can forward the alcoholic liquid, e.g. cyclically, through the conduit(s) of the clamp plate. The alcoholic liquid tank advantageously is filled with an alcoholic liquid such as a pure alcohol, an alcohol solution or a mixture comprising alcohol. Advantageously the alcoholic liquid is Ethanol.

Preferably, the plate cleaning device comprises a pump configured to forward the first fluid and the second fluid from the first fluid terminal and the second fluid terminal to each of the valve units. Such pump allows for accurately and efficiently provide the first and second fluids for cleaning the clamp plate.

The term “pump” as used in this connection may relate to any means suitable for a controlled forwarding of the respective fluid. Thereby, the involved fluid may be actively forwarded such as by the pump creating an overpressure in the fluid container or an under-pressure to withdraw the fluid from the fluid container. Or, the pump may be a passive means such as a valve of a pressurized fluid container allowing to adjust a fluid flow out of the container.

Thereby, the control unit preferably is configured to control the pump such the that first fluid and the second fluid are selectively forwardable to the valve units. Like this, the control unit may be embodied to apply sophisticated cleaning and drying according to a pre-defined or adjusted plan or scheme.

Preferably, the plate cleaning device comprises a pressurized gas vessel coupled to each of the valve units, wherein the control unit is configured to control the valve units such that the pressurized gas vessel together with the first fluid terminal and the second fluid terminal are selectively connected to the second open ends of the channels of the cover member. The pressurized gas can be pressurized air or a similar gas. Like this, the control unit may provide the gas through the clamp plate and particularly through its conduit(s). For example, this allows for removing solid or liquid residuals from the conduit(s). More specifically, such gas provision allows for drying the conduit(s).

Preferably, the plate cleaning device comprises a cleanness evaluation arrangement to assess a level of cleanness of the conduit(s) of the clamp plate. By means of such cleanness evaluation arrangement the cleanness of the conduit(s) can be determined. Like this, it can be achieved that the clamp plate is processed until an aimed level of cleanness is achieved.

Thereby, the cleanness evaluation arrangement preferably comprises a flow meter configured to measure a gas flow inside each of the conduit(s) of the clamp plate, when the plate seat member holds the clamp plate and the cover member closes the plate seat member. Such flow meter allows for efficiently determining if any residuals are in the conduits of the clamp plates. In particular, it can be determined conduit per conduit if a sufficient cleanness is achieved. More specifically, at a given gas pressure the dimension of the flow of gas through the conduit is an indication if the conduit is free and, thus, does not contain any residual solid or liquid substance. Also, the dimension of the gas flow through or permeability of the conduits allows for providing or concluding information about a level of cleanness of each of the conduits.

Preferably, the control unit is configured to apply a cleaning procedure involving at least a step of forwarding the first fluid though the conduits of the clamp plate and a step of forwarding the second fluid through the conduits of the clamp plate. In particular, the cleaning procedure can be set up by a cleaning scheme defining parameters of the provision of the first and second fluids through the conduit(s). Such parameters may include duration, pressure and others. The control unit may also be configured to allow an operator to predefine or set up the cleaning procedure or cleaning scheme. Like this, the clamp cleaning device can be adjusted in accordance with the particular clamp plate and substances involved in the experiments using the clamp plate.

Thereby, the control unit preferably is configured to apply the cleaning procedure with an additional step of forwarding gas through the conduit(s) of the clamp plate. Like this, the conduit(s) can be dried and/or any liquid or loose solid residual can be removed out of the conduit(s). Further, the cleanness evaluation arrangement or the flow meter thereof can measure gas flow through the conduit(s). Like this, a level of cleanness can be determined.

In a preferred embodiment, the control unit is configured to repeat at least a portion of the cleaning procedure if the gas flow measured by the flow meter is below a predefined threshold. Thereby, the term “configured to repeat” may include that the control unit automatically repeats the cleaning procedure if the gas flow is below the predefined threshold, or that the control unit is manually triggered, e.g. by pushing a button, to repeat the cleaning procedure. In the second case, the control unit may provide an information about the cleanness of the conduits or provide a signal that the cleanness of at least some of the conduits is not sufficient.

The term “repeat at least a portion of the cleaning procedure” covers that all or some of the steps of the cleaning procedure are performed and/or that all or some of the conduits undergo the cleaning procedure. In particular, this term may relate to only providing the cleaning procedure to the conduits in which the gas flow measured by the flow meter is below the predefined threshold.

In particular, since the gas flow may be indicative for a level of cleanness inside the conduit(s) a measured gas flow being too low allows to determine an insufficient cleanness level. As a consequence, the provision of the cleaning fluids or the cleaning procedure is repeated to increase the level of cleanness and, thus, the gas flow through the conduit(s).

Preferably, the plate seat member is equipped with a fluid outlet. Such fluid outlet allows for providing fluids used for cleaning the clamp plate out of the plate cleaning device.

Preferably, the plate cleaning device comprises a fixation formation configured to be arranged in a locked position in which it presses the cover member to the clamp plate, when the plate seat member holds the clamp plate and the cover member closes the plate seat member, and in an unlocked position in which the cover member is releasable from the plate seat member. Such fixation formation allows to efficiently enclosing the clamp plate and ensuring tightness during the cleaning procedure.

Thereby, the fixation formation preferably comprises an actuation member to switch between the locked position and the unlocked position. Such actuation member allows for efficiently closing and opening the device, e.g. manually.

Preferably, the control unit is coupled to the valve units and configured to modulate the valve units in order to selectively connect the first fluid terminal and the second fluid terminal to the second open ends of the channels of the cover member. By such design the control unit can efficiently implement a sophisticated cleaning procedure.

### BRIEF DESCRIPTION OF THE DRAWINGS

The plate cleaning device according to the invention is described in more detail hereinbelow by way of an exemplary embodiment and with reference to the attached drawings, in which:

FIG. 1 shows a top view of a clamp plate;

FIG. 2 shows a top view of a cover plate of an embodiment of a plate cleaning device according to the invention;

FIG. 3 shows an exploded perspective view of the clamp plate of FIG. 1 as well as the cover plate, a valve and a base of the plate cleaning device of FIG. 2;

FIG. 4 shows a side view of a water tank as example of a pressurized liquid tank of the plate cleaning device according to the invention; and

FIG. 5 shows a side view of the plate cleaning device of FIG. 2.

### DESCRIPTION OF EMBODIMENTS

In the following description certain terms are used for reasons of convenience and are not intended to limit the invention. The terms “right”, “left”, “up”, “down”, “under” and “above” refer to directions in the figures. The terminology comprises the explicitly mentioned terms as well as their derivations and terms with a similar meaning. Also, spatially relative terms, such as “beneath”, “below”, “lower”, “above”, “upper”, “proximal”, “distal”, and the like, may be used to describe one element's or feature's relationship to another element or feature as illustrated in the figures. These spatially relative terms are intended to encompass different positions and orientations of the devices in use or operation in addition to the position and orientation shown in the figures. For example, if a device in the figures is turned over, elements described as “below” or “beneath” other elements or features would then be “above” or “over” the other elements or features. Thus, the exemplary term “below” can encompass both positions and orientations of above and below. The devices may be otherwise oriented (rotated 90 degrees or at other orientations), and the spatially relative descriptors used herein interpreted accordingly. Likewise, descriptions of movement along and around various axes include various special device positions and orientations.

To avoid repetition in the figures and the descriptions of the various aspects and illustrative embodiments, it should be understood that many features are common to many aspects and embodiments. Omission of an aspect from a description or figure does not imply that the aspect is missing from embodiments that incorporate that aspect. Instead, the aspect may have been omitted for clarity and to avoid prolix description. In this context, the following applies to the rest of this description: If, in order to clarify the drawings, a figure contains reference signs which are not explained in the directly associated part of the description, then it is referred to previous or following description sections. Further, for reason of lucidity, if in a drawing not all features of a part are provided with reference signs it is referred to other drawings showing the same part. Like numbers in two or more figures represent the same or similar elements.

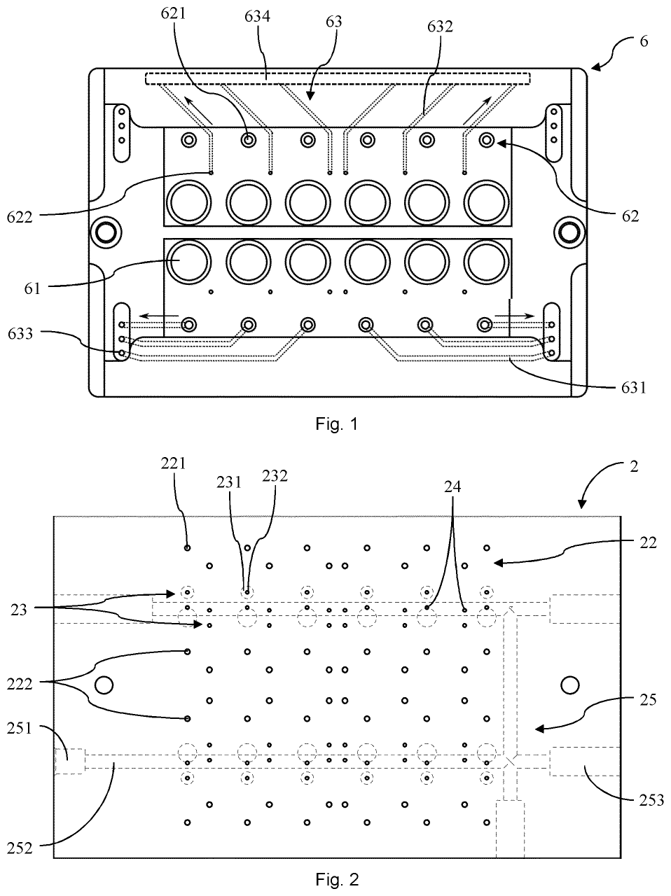

FIG. 1 shows a clamp plate 6 suitable to be processed or cleaned in an embodiment of a plate cleaning device 1 according to the invention. The clamp plate 6 is a type 001-0B38 clamp plate of a microbioreactor system Ambr® 15 of Sartorius Stedim Biotech GmbH. It comprises two rows of six receptacles 61 each embodied to receive a disposable bioreactor. Each receptacle 61 is associated to two conduit openings 62 of internal conduits 63 embodied inside the clamp plate 6. In particular, the clamp plate 6 comprises twelve lateral conduits 632 (in FIG. 1 only the upper six lateral conduits 632 are depicted) and twelve longitudinal conduits 631 (in FIG. 1 only the lower six longitudinal conduits 631 are depicted).

Each lateral conduit 632 has a fluid inlet opening 622 as conduit opening 62 at a top face of the clamp plate 6. The lateral conduits 632 are arranged in two groups of six lateral conduits 632, wherein each group of lateral conduits 632 has a common fluid outlet 634 at the bottom face of the clamp plate 6. Each longitudinal conduit 631 has a fluid inlet opening 621 as conduit opening 62 at the top face of the clamp plate 6 and a fluid outlet opening 633 at the top face of the clamp plate 6. The lateral conduits 632 and their inlet openings 622 are smaller in dimension as the longitudinal conduits 631 and their inlet openings 621. More specifically, the clamp plate 6 provides one fluid inlet opening 622 of one lateral conduit 632 and one fluid inlet opening 621 of one longitudinal conduit 631 per receptacle 61.

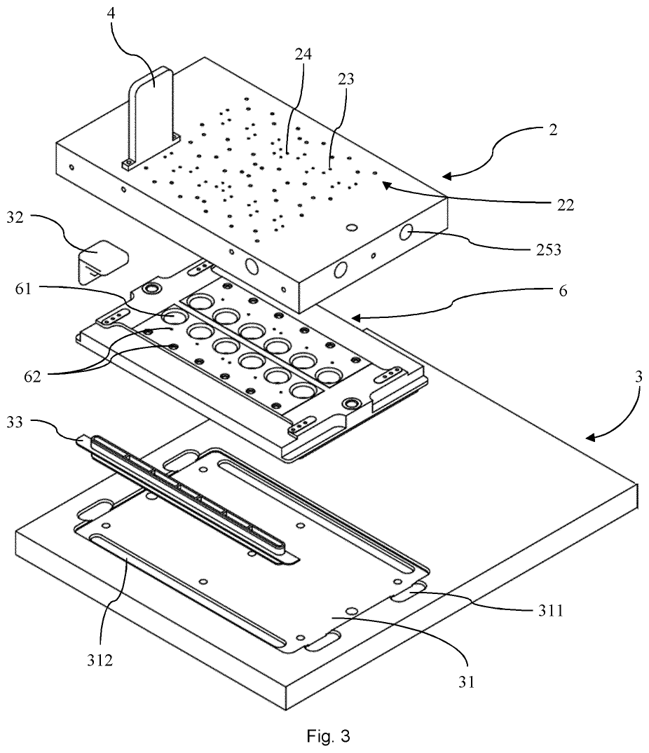

In FIG. 2 a cover plate 2 as cover member of the plate cleaning device 1 is shown. The cover plate 2 has an internal fluid channel system 25 indicated in FIG. 2 by dotted lines. In particular, the fluid channel system 25 comprises a fluid inlet 251 at a left side edge of the cover plate 2 from which a main channel 252 extends parallel to a lower longitudinal side edge of the cover plate 2 to a right side edge of the cover plate 2 where it ends in a capped end 253. Parallel to the right side edge of the cover plate 2 the main channel 252 extends from a capped end 253 in the lower longitudinal side edge of the cover plate 2 towards an upper longitudinal side edge of the cover plate 2 and parallel to the upper longitudinal side edge of the cover plate 2 the main channel 252 extends from a capped end 253 in the right side edge of the cover plate 2 towards a capped end 253 in the left side edge of the cover plate 2. Like this, the main channel 252 openly extends from the lower left end side edge parallel to the lower longitudinal side edge, turns by 90° to extend parallel towards the upper longitudinal side edge, turns again by 90° to extend parallel the upper longitudinal side edge towards the left side edge.

The cover plate 2 further comprises a plurality of fixation drills 22 provided in the top face of the cover plate 2. In particular, the fixation drills 22 comprise twelve peripheral fixation drills 221 along the upper longitudinal side of the cover plate 2, corresponding twelve peripheral fixation drills 221 along the lower longitudinal side of the cover plate 2 and twenty-four central fixation drills 222.

Further, the cover plate 2 is equipped with twenty-four through bores 23 as straight channels, each extending from a lower first open end 231 at the bottom face of the cover plate 2 to an upper second open end 232 at the top face of the cover plate 2. The through bores 23 are positioned such each first open end 231 of one through bore 23 is connected to one of the conduit openings 62 of the clamp plate 6, when the cover plate 2 appropriately covers the clamp plate 6. Still further, the cover plate 2 is equipped with twenty-four straight channel bores 24 extending from the top face of cover plate 2 to the main channel 252 of the fluid channel system 25.

FIG. 3 shows the cover plate 2, one valve 4 as valve unit of a plurality of valve units and a base 3 as plate seat member of the plate cleaning device 1 according to the invention together with the clamp plate 6 to be cleaned.

The base 3 has a plate seat 31 designed to receive and hold the clamp plate 6 in a predefined position and orientation. It further comprises lateral rerouting pieces 32 and longitudinal rerouting pieces 33. The lateral and longitudinal rerouting pieces 32, 33 are inserted into corresponding lateral outlets 311 and longitudinal outlets 312 embodied in the plate seat 31 of the base 3. More specifically, when the clamp plate 6 is arranged in the plate seat 31, the longitudinal rerouting pieces 33 are positioned under and along the clamp plate 6 such that they are coupled to the fluid outlets 634 at the bottom face of the clamp plate 6. The lateral rerouting pieces 32 connect openings at a top side of the clamp plate 6 with the lateral outlets 34 of the base 3.

The cover plate 2 is designed to cover the clamp plate 6 when the latter is received by the plate seat 31 of the base 3. Thereby, the clamp plate 6 is tightly enclosed by the base 3 and the cover plate 2, when the base 3 holds the clamp plate 6 in the plate seat 31 and the cover plate 2 closes the plate seat 31. More specifically, each through bore 23 of the cover plate 2 is connected to one of the fluid inlet openings 621, 622 of the longitudinal conduits 631 and the lateral conduits 632 of the clamp plate 6.

The valve 4 is configured to be coupled to the top face of the cover plate 2 by being fixed to a pair of fixations drills 22 consisting of one lateral fixation drill 221 and one central fixation drill 222. When being fixed to the cover plate 2, the valve 4 couples one of the through bores 23 and one of the channel bores 24. In particular, the valve 4 is arranged to selectively connect and disconnect the associated through bore 23 and channel bore 24. In operation of the plate cleaning device 1, the plurality of valves 4 is fixed to the cover plate 2 such that one valve 4 connected to each through bore 23 and channel bore 24 of the cover plate 2 such that one valve 4 is associated to each of the conduit openings 62 of the clamp plate 6. When the valve 4 connects the associated through bore 23 and channel bore 24, the associated conduit opening is in fluid connection with the main channel 252 of the fluid channel system 25 of the clamp plate 5. Like this, a fluid can be provided from the main channel 252 into the longitudinal or lateral conduit 631, 632 in a cleaning procedure.

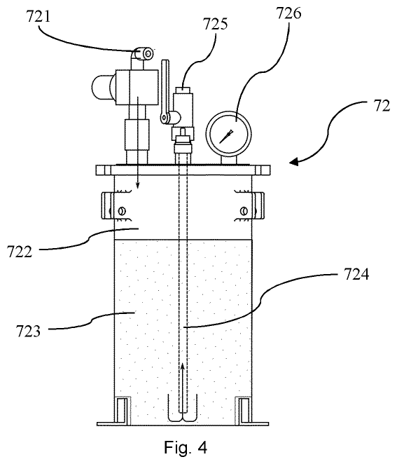

In FIG. 4 an example of a pressurized water tank 72 filled with purified water 723 is shown. The water tank 72 has an air inlet 721 through which pressurized air can be provided into the interior of the water tank 72. In particular, by providing pressurized air into the water tank 72, the interior of the water tank 72 is pressurized as well such that the purified water is upwardly forwarded into an outlet pipe 724. At its top end, the outlet pipe 724 passes over into a liquid outlet 725 provided with an actuator for opening and closing the liquid outlet. The water tank 72 further is equipped with a pressure sensor 726 indicating the pressure in the interior of the water tank 72.

Even though FIG. 4 exemplarily shows a pressurized water tank 72 the same structure can also be used for liquids. In particular, the same structure is also used as pressurized Ethanol tank 75 as described below.

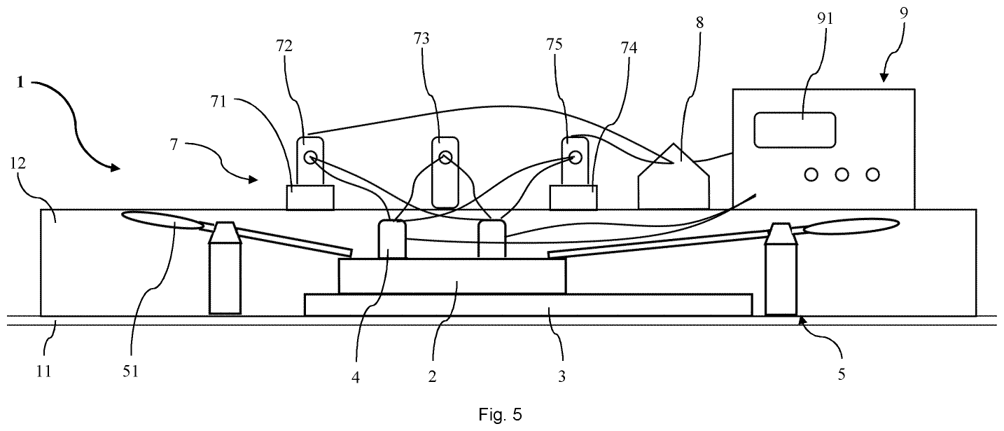

In FIG. 5, the plate cleaning device 1 is shown in an assembled state ready to be operated. The base 3 together with the cover plate 2 is positioned on a tray 11 which carries a platform 12. Adjacent to each longitudinal side of the base plate 3 a press fixator 5 is positioned as fixation formation on the tray 11. The press fixators 5 each have a pillar to which an actuation member 51 with a handle is mounted. The actuation members 51 are designed to be arranged in a locked position as shown in FIG. 2, in which they press the cover plate 2 to the base 3, and in an unlocked position in which the cover plate 2 is releasable from the base 3. In the locked position, the press fixators 5 assure that the cover plate 2 and the base 3 tightly enclose the clamp plate 6.

On the platform 12, a tank equipment 7 is arranged. The tank equipment 7 comprises a water tank terminal 71 as first fluid terminal and an Ethanol tank terminal 74 as second fluid terminal. Between the water tank terminal 71 and the Ethanol tank terminal 74 an air bottle 73 is positioned as pressurized gas vessel. The water tank terminal 71 receives the water tank 72 described above. The Ethanol tank terminal 74 receives the Ethanol tank 75 filled with Ethanol. The air bottle 73 is filled with pressurized air as pressurized gas. The water tank 72, the air bottle 73 and the Ethanol tank 75 are connected to the fluid inlet 251 of the cover plate 2.

The water tank 72 and the Ethanol tank 75 may additionally be coupled to an optional pump 8 which is configured to forward the purified water and Ethanol to the valves 4. The plate cleaning device 1 further comprises a control unit 9 which is coupled to the pump 8 and each of the valves 4. The control unit 9 has a display 91 as integrated user interface and some buttons for basic operation and configuration. Further, the control unit 9 is configured to be accessed by a laptop computer by wireless communication or by other digital communication channel in order to configure operation parameters. After configuring, the laptop computer is not required in operation of the plate cleaning device 1.

In operation, the control unit 9 is preconfigured by the operator of the system. In particular, parameters defining a cleaning protocol or scheme are set in the control unit 9. The control unit 9 then activates the valves 4 such that purified water, Ethanol and air selectively and cyclically is provided through the conduits 62 of the clamp plate 6 via the fluid channel system 25 of the cover plate 2. More specifically, the conduits 63 are flushed with the purified water and Ethanol, and dried with the air in accordance with the cleaning scheme. After passing the conduits 63, the water and Ethanol are routed by the lateral and longitudinal rerouting pieces 32, 33 into the outlets 311, 312.

The plate cleaning device 1 further comprises a flow meter (not visible in FIG. 2) which is coupled to the control unit 9 and configured to measure an air flow through each of the conduits 63 of the clamp plate 6. After performing the procedure defined by the cleaning scheme, the control unit 9 provides air through the conduits 63 and evaluates the signals received from the flow meter. In particular, the control unit 9 compares the measured flow in the conduits 63 to a predefined threshold value. If the measured flow is below the threshold value, the control unit 9 initiates further cleaning of the conduits 63 by forwarding purified water, Ethanol and air through the conduits 63. Also, in such case the affected conduit(s) are displayed to an operator of the plate cleaning device 1. Only, when successfully achieving the predefined threshold flow in the conduits 63 the control unit 9 will indicate the clamp plate 6 to be at an appropriate cleanness level. If an aimed cleanness cannot be achieved the respective clamp plate 6 can be replaced by a new one.

This description and the accompanying drawings that illustrate aspects and embodiments of the present invention should not be taken as limiting—the claims defining the protected invention. In other words, while the invention has been illustrated and described in detail in the drawings and foregoing description, such illustration and description are to be considered illustrative or exemplary and not restrictive. Various mechanical, compositional, structural, electrical, and operational changes may be made without departing from the spirit and scope of this description and the claims. In some instances, well-known circuits, structures and techniques have not been shown in detail in order not to obscure the invention. Thus, it will be understood that changes and modifications may be made by those of ordinary skill within the scope and spirit of the following claims. In particular, the present invention covers further embodiments with any combination of features from different embodiments described above and below

The disclosure also covers all further features shown in the Figs. individually although they may not have been described in the afore or following description. Also, single alternatives of the embodiments described in the figures and the description and single alternatives of features thereof can be disclaimed from the subject matter of the invention or from disclosed subject matter. The disclosure comprises subject matter consisting of the features defined in the claims or the exemplary embodiments as well as subject matter comprising said features.

Furthermore, in the claims the word “comprising” does not exclude other elements or steps, and the indefinite article “a” or “an” does not exclude a plurality. A single unit or step may fulfil the functions of several features recited in the claims. The mere fact that certain measures are recited in mutually different dependent claims does not indicate that a combination of these measures cannot be used to advantage. The terms “essentially”, “about”, “approximately” and the like in connection with an attribute or a value particularly also define exactly the attribute or exactly the value, respectively. The term “about” in the context of a given numerate value or range refers to a value or range that is, e.g., within 20%, within 10%, within 5%, or within 2% of the given value or range. Components described as coupled or connected may be electrically or mechanically directly coupled, or they may be indirectly coupled via one or more intermediate components. Any reference signs in the claims should not be construed as limiting the scope.

1. A plate cleaning device for cleaning a clamp plate of a fermenter, wherein the clamp plate has a plurality of receptacles each configured to hold a bioreactor vessel, and at least one conduit with a conduit opening per receptacle, comprising:
a plate seat member configured to hold the clamp plate in a predefined position and orientation;
a cover member configured to close the plate seat member such that the clamp plate is tightly enclosed by the plate seat member and the cover member, when the plate seat member holds the clamp plate and the cover member closes the plate seat member;
a plurality of valve units configured to be coupled to the cover member such that at least one valve unit is associated to each of the conduit openings of the clamp plate, when the plate seat member holds the clamp plate and the cover member closes the plate seat member;
a first fluid terminal configured to receive a first fluid container;
a second fluid terminal configured to receive a second fluid container; and
a control unit, wherein
the cover member is equipped with channels each having a first open end and a second open end,
each of the first open ends of the channels of the cover member is positioned to be connected to one of the conduit openings of the clamp plate, when the plate seat member holds the clamp plate and the cover member closes the plate seat member,
the valve units are configured to be coupled to the second open ends of the channels of the clamp plate such that all of the at least one conduit openings per receptacle are associated to one valve,
the first fluid terminal and the second fluid terminal are coupled to each of the valve units, and
the control unit is configured to selectively connect the first fluid terminal and the second fluid terminal to the second open ends of the channels of the cover member.

2. The plate cleaning device of claim 1, comprising a water tank as the first fluid container, wherein the water tank is received in the first fluid terminal.

3. The plate cleaning device of claim 1, comprising an alcoholic liquid tank as the second fluid container, wherein the alcoholic liquid tank is received in the second fluid terminal.

4. The plate cleaning device of claim 1, comprising a pump configured to forward a first fluid from the first fluid terminal and a second fluid from the second fluid terminal to each of the valve units.

5. The plate cleaning device of claim 4, wherein the control unit is configured to control the pump such that the first fluid and the second fluid are selectively forwardable to the valve units.

6. The plate cleaning device of claim 1, comprising a pressurized gas vessel coupled to each of the valve units, wherein the control unit is configured to control the valve units such that the pressurized gas vessel together with the first fluid terminal and the second fluid terminal are selectively connected to the second open ends of the channels of the cover member.

7. The plate cleaning device of claim 1, comprising a cleanness evaluation arrangement to assess a level of cleanness of the conduits of the clamp plate (6).

8. The plate cleaning device of claim 7, wherein the cleanness evaluation arrangement comprises a flow meter configured to measure a gas flow through each of the conduits of the clamp plate, when the plate seat member holds the clamp plate and the cover member closes the plate seat member.

9. The plate cleaning device of claim 1, wherein the control unit is configured to apply a cleaning procedure involving at least a step of forwarding a first fluid though the conduits of the clamp plate and a step of forwarding a second fluid through the conduits of the clamp plate.

10. The plate cleaning device of claim 9, wherein the control unit is configured to apply the cleaning procedure with an additional step of forwarding gas through the conduits of the clamp plate.

11. The plate cleaning device of claim 10, wherein a cleanness evaluation arrangement comprises a flow meter configured to measure a gas flow through each of the conduits of the clamp plate, when the plate seat member holds the clamp plate and the cover member closes the plate seat member, and wherein the control unit is configured to repeat at least a portion of the cleaning procedure if the gas flow measured by the flow meter is below a predefined threshold.

12. The plate cleaning device of claim 1, wherein the plate seat member is equipped with a fluid outlet.

13. The plate cleaning device of claim 1, comprising a fixation formation configured to be arranged in a locked position in which it presses the cover member to the clamp plate, when the plate seat member holds the clamp plate and the cover member closes the plate seat member, and in an unlocked position in which the cover member is releasable from the plate seat member.

14. The plate cleaning device of claim 13, wherein the fixation formation comprises an actuation member to switch between the locked position and the unlocked position.

15. The plate cleaning device of claim 1, wherein the control unit is coupled to the valve units and configured to modulate the valve units in order to selectively connect the first fluid terminal and the second fluid terminal to the second open ends of the channels of the cover member.
